(19) United States
(12) Patent Application Publication
(10) Pub. No.: US 2011/0124935 A1
(43) Pub. Date: May 26, 2011

(54) PROCESS FOR PRODUCING AROMATIC COMPOUND

(75) Inventor: Takuya Hatagishi, Shizuoka (JP)

(73) Assignee: MEIDENSHA CORPORATION

(21) Appl. No.: 13/055,611

(22) PCT Filed: May 19, 2009
(86) PCT No.: PCT/JP2009/059153

§ 371 Date: Jan. 24, 2011

(30) Foreign Application Priority Data
Jul. 29, 2008 (JP) 2008-194392

(51) Int. Cl. C07C 2/76

(52) U.S. Cl. 585/433; 585/430

(57) ABSTRACT

[Task] In a method for producing an aromatic compound by a catalytic reaction using a lower hydrocarbon as a raw material, yields of hydrogen and the aromatic compound are to be improved, and a stable catalytic activity is to be maintained.

[Solving Means] Molybdenum or a molybdenum compound is supported on metallosilicate, and then a carbonization treatment is conducted, thereby obtaining a lower-hydrocarbon aromatizing catalyst. A reaction gas containing a lower hydrocarbon is brought into contact with this catalyst, thereby producing an aromatic compound. Upon this, while allowing a non-oxidizing gas (except hydrocarbon gas) to flow, a temperature rising is conducted to a catalytic reaction temperature. When it reaches the catalytic reaction temperature, the reaction gas is allowed to flow, and the reaction gas is brought into contact with the catalyst, thereby obtaining an aromatic compound such as benzene or naphthalene.

### TECHNICAL FIELD

The present invention relates to a high-degree use of natural gas, biogas and methane hydrate, in which methane is a main component. Natural gas, biogas, and methane hydrate are regarded as the most effective energy resources as global warming measures, and an interest in its use technique is increasing. Methane resource making use of its clean property attracts an attention as the next generation new organic resource and as a hydrogen resource for fuel cells. In particular, the present invention relates to a catalytic chemical conversion technique for effectively producing aromatic compounds, in which benzene and naphthalenes, which are raw materials of chemical products such as plastics, are main components, and a high-purity hydrogen gas, from methane.

### BACKGROUND TECHNIQUE

As a process for producing hydrogen and aromatic compounds, such as benzene, from methane, one is known in which methane is reacted in the presence of a catalyst. As the catalyst upon this, molybdenum supported on a ZSM-5 series zeolite is said to be effective (Non-patent Publication 1). However, even in the case of using these catalysts, there are problems that carbon is precipitated in large amount and that conversion of methane is low.

In order to solve this problem, there has been proposed a catalyst in which a catalyst material such as Mo (molybdenum) is supported on a porous metallosilicate, as disclosed, for example, in Patent Publication 1 to Patent Publication 3. It has been confirmed in Patent Publication 1 to Patent Publication 3 that lower hydrocarbons are efficiently turned into aromatic compounds by using a catalyst in which a metal component is supported on a porous metallosilicate as a support having a micropore diameter of 7 angstroms, and along with this a high purity hydrogen is obtained.

Then, in Patent Publication 4 to Patent Publication 6, molybdenum is subjected to carbonization treatment by treating a molybdenum-supported metallosilicate with a mixed gas of methane and hydrogen. That is, a molybdenum-supported catalyst is subjected to carbonization treatment, thereby stabilizing and improving the rate of forming aromatic compounds and hydrogen.

### Prior Art Publications

### Patent Publications

Patent Publication 1: Japanese Patent Application Publication No. 2004-91891

### Patent Publication 2: Japanese Patent No. 3755955

### Patent Publication 3: Japanese Patent No. 3745885

### Patent Publication 4: International Application Publication 2005/028105 Pamphlet

### Patent Publication 5: Japanese Patent No. 3835765

### Patent Publication 6: Japanese Patent Application Publication No. 2005-254122

### Non-Patent Publications

Non-patent Publication 1: JOURNAL OF CATALYSIS, 1997, p. 165 and p. 150-161

### SUMMARY OF THE INVENTION

### Task to be Solved by the Invention

However, in the above-mentioned conventional techniques, there are problems that catalytic performance is deteriorated in a short period of time by carbon precipitation, that conversion of methane is low, and the like. Thus, there is a demand for the development of a more superior catalyst.

Furthermore, in the conventional techniques shown in the above Patent Publication 4 to Patent Publication 6, when it is raised until the catalytic reaction temperature after the carbonization treatment, it is raised until the catalytic reaction temperature under an atmosphere of a gas used in the carbonization treatment or of a gas to be used for the catalytic reaction.

A hydrocarbon gas such as methane is contained in the gas used in the gas carbonization treatment and the gas to be used for the catalytic reaction. When the temperature is raised until the catalytic reaction temperature under an atmosphere containing such hydrocarbon gas, there is a fear that a large amount of coke is precipitated to interfere with the catalytic reaction.

It is therefore an object of the present invention to provide a method for further increasing the efficiency of producing aromatic compounds and hydrogen in an aromatic compound production method for producing aromatic compounds by a catalytic reaction using a lower hydrocarbon as the raw material.

### Means for Solving the Task

An aromatic compound production method of the present invention, in which a lower hydrocarbon is used as the raw material, for achieving the object is a method for producing aromatic compounds by a catalytic reaction using a lower hydrocarbon as the raw material and is characterized in that a catalyst used in the catalytic reaction is subjected to a temperature rising until a catalytic reaction temperature under an atmosphere of a non-oxidizing gas (except hydrocarbon gas) and that a gas containing a lower hydrocarbon is brought into contact with the catalyst to produce aromatic compounds.

Furthermore, the non-oxidizing gas is characterized by being a reducing gas or an inert gas. Herein, the reducing gas is exemplified by hydrogen, carbon monoxide, and ammonia. Then, the inert gas is exemplified by argon, nitrogen, and helium.

Then, the catalyst is characterized by being a catalyst prepared by supporting molybdenum or a molybdenum compound on a metallosilicate and then conducting a carbonization treatment.

According to the above-mentioned aromatic compound production method, it is possible to raise the temperature until an optimum catalytic reaction temperature without damaging activity of the catalyst.

### ADVANTAGEOUS EFFECT OF THE INVENTION

Therefore, according to the above-mentioned invention, in a method for producing aromatic compounds by a catalytic reaction using a lower hydrocarbon as the raw material, yields of hydrogen and the aromatic compounds are improved, and the catalyst activity lifetime stability is improved.

### BRIEF DESCRIPTION OF THE DRAWINGS

FIG. 1 shows a change over time of methane conversion in the case of reacting each catalyst of Comparative Example 1, Comparative Example 2 and Example 1 with a carbonic acid gas-mixed methane gas (the molar ratio of methane to carbonic acid gas, methane:carbonic acid gas (carbon dioxide), is 20:1);

FIG. 2 shows a change over time of benzene conversion in the case of reacting each catalyst of Comparative Example 1, Comparative Example 2 and Example 1 with a carbonic acid gas-mixed methane gas (the molar ratio of methane to carbonic acid gas, methane:carbonic acid gas (carbon dioxide), is 20:1);

FIG. 3 shows a change over time of naphthalene conversion in the case of reacting each catalyst of Comparative Example 1, Comparative Example 2 and Example 1 with a carbonic acid gas-mixed methane gas (the molar ratio of methane to carbonic acid gas, methane:carbonic acid gas (carbon dioxide), is 20:1);

FIG. 4 shows a change over time of BTX conversion in the case of reacting each catalyst of Comparative Example 1, Comparative Example 2 and Example 1 with a carbonic acid gas-mixed methane gas (the molar ratio of methane to carbonic acid gas, methane:carbonic acid gas (carbon dioxide), is 20:1); and

FIG. 5 shows a schematic diagram of a fixed bed, flow type reaction apparatus used for aromatizing a lower hydrocarbon, according to the present invention.

### MODE FOR CARRYING OUT THE INVENTION

A catalyst for aromatizing a lower hydrocarbon according to an embodiment of the present invention contains as a catalyst material at least one type selected from molybdenum and its compounds. When aromatic compounds are produced, the catalyst for aromatizing a lower hydrocarbon is reacted with carbon dioxide besides the lower hydrocarbon.

A support for supporting the metal component contains a porous metallosilicate having micropores that are substantially 4.5 to 6.5 angstroms in diameter. Detailed explanations such as the type of metallosilicate used are described in Patent Publication 1 (Japanese Patent Application Publication 2004-91891), which is a conventional technique.

As to the molybdenum component, the metallosilicate is added to an impregnation aqueous solution prepared with ammonium molybdate. Thus, the metallosilicate is impregnated with the molybdenum component, and then it is subjected to drying and baking. With this, the molybdenum component is supported on the metallosilicate.

The molybdenum component-supported metallosilicate is subjected to temperature rising until a predetermined temperature under an atmosphere of a mixed gas of methane and hydrogen, and is maintained for a predetermined time, thereby conducting a carbonization treatment of the catalyst.

It is possible to obtain stability of the catalyst by subjecting the catalyst of after the carbonization treatment to a temperature rising until the catalytic reaction temperature with a non-oxidizing gas (e.g., N₂, Ar, He, etc.). In particular, there improve stabilities over time of methane conversion, benzene yield, naphthalene yield, and BTX yield (the total yield of benzene, toluene and xylene).

To produce aromatic compounds, a reaction gas containing a lower hydrocarbon and carbon dioxide is reacted with the lower-hydrocarbon aromatizing catalyst. The amount of the carbon dioxide to be added is set, for example, in a range of 0.5-6% relative to the total of the reaction gas.

Based on the following comparative examples and example, the lower-hydrocarbon aromatizing catalyst of the present invention is explained.

### Comparative Example 1

### 1. Production of Lower-Hydrocarbon Aromatizing Catalyst (in the Following, Abbreviated as Catalyst)

In the catalyst of Comparative Example 1, ammonium-type ZSM-5 (SiO₂/Al₂O₃=25-70) was used as the metallosilicate, and it is one having molybdenum supported on this.

(1) Combination

Combination of inorganic components: ZSM-5 (82.5 weight %), clay (12.5 weight %), and glass fibers (5 weight %).

Total Combination: the inorganic components (76.5 weight %), organic binder (17.3 weight %), and water (24.3 weight %)

(2) Molding

The inorganic components, organic binder, and water were combined at the combination ratio, followed by mixing and kneading by a kneading means (kneader). Then, this mixture was molded into a rod shape (diameter 2.4 mm×length 5 mm) by a vacuum extruder. The extrusion pressure upon this molding was set at 2-8 MPa.

In general, the catalyst support used for reforming hydrocarbons is used as a flow bed catalyst using particles having a particle diameter of from several micrometers to several hundreds micrometers. In the production method of the catalyst support of this case, a support material of the catalyst is mixed with organic binder, inorganic binder (normally clay is used), and water to make a slurry form, followed by granulation molding (there is no molding pressure) and then baking. In this case, due to no molding pressure, the amount of clay added as a baking assistant in order to assure the baking speed was about 40-60 wt %. Herein, molding of the catalyst is conducted by a high-pressure molding using a vacuum extruder. With this, it is possible to reduce the amount of additives, such as clay, added as a baking assistant to 15-25 wt %. Therefore, it is possible to improve the catalyst activity.

(3) Impregnation of Molybdenum

An impregnation aqueous solution prepared by ammonium molybdate was stirred. To the impregnation aqueous solution under this stirring condition, a ZSM-5 containing mold after the molding step was added to impregnate the mold with the molybdenum component. Then, it was subjected to the following drying and baking steps. In the preparation of the impregnation aqueous solution, the amount of molybdenum to be supported was set at 6 wt % relative to the total amount of the catalyst after the baking.

(4) Drying & Baking

In the drying step, a drying was conducted at 70° C. for about 12 hours in order to remove water added in the molding step, and then a drying was conducted at 90° C. for 36 hours. In the baking step, a baking was conducted at 550° C. for 5 hours in the air. The baking temperature in the baking step was set in a range of 550-800° C., due to lowering of strength of the support if it is lower than 550° C. and due to lowering of properties (activity) if it is higher than 800° C. The rate of temperature increase and the rate of temperature decrease in the baking step were set at 90-100° C./hour. Upon this, in order to prevent an instantaneous combustion of the organic binder added in the molding, a temperature keeping for about 2 to 6 hours in a temperature range of 250-500° C. was conducted two times to remove the binder. It is because an instantaneous combustion of the binder occurs to lower strength of the baked body in case that the rate of temperature increase and the rate of temperature decrease are greater than the above rate and that the keeping time for removing the binder is not taken.

(5) Carbonization Treatment

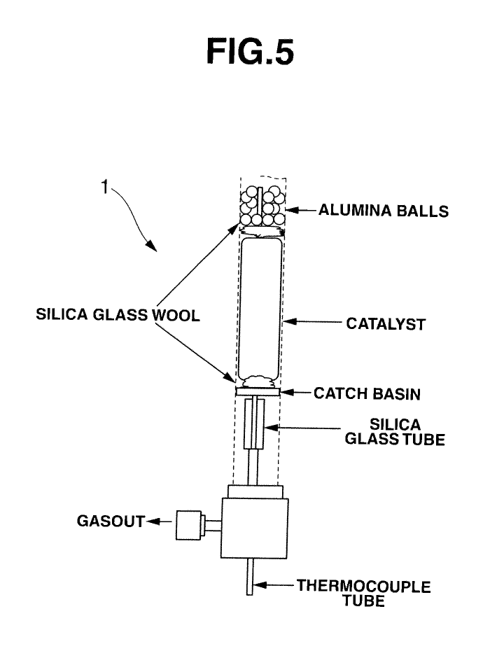

A reaction tube (inner diameter 18 mm) made by an Inconel 800H, gas-contact portion calorizing treatment of a fixed bed flow-type reaction apparatus 1 shown in FIG. 5 was charged with the baked body. Then, the baked body was subjected to a temperature rising until 700° C. by 2 hours under a flow (flow rate 0.5 L/min) of a mixed gas of CH₄ and H₂ (methane (mol): hydrogen (mol)=1:4). This condition was maintained for 3 hours. Herein, the carbonization of molybdenum is preferably from 600 to 750° C. This is because it is inefficient by a considerable lowering of the rate of carbonization of molybdenum if it is lower than 600° C., and because a coke precipitation reaction is accelerated if it is higher than 750° C.

(6) Temperature Rising toward Catalytic Reaction Temperature

After the carbonization treatment of the baked body, a temperature rising was conducted until 800° C. by 10 minutes, while supplying a CH₄ reaction gas (methane (mol) carbon dioxide (mol)=20:1) to the reaction tube shown in FIG. 5.

### Comparative Example 2

The catalyst of Comparative Example 2 is the same as that of Comparative Example 1 in terms of combination and production method, except the temperature rising condition toward the catalytic reaction temperature. That is, the catalyst was produced by the same method as the combination and the production steps of Comparative Example 1. Then, the reaction tube was charged therewith, followed by the carbonization treatment and then a temperature rising until 800° C. by 15 minutes while supplying a CH₄ reaction gas (methane (mol) carbon dioxide (mol)=20:1) to the reaction tube.

### Example 1

The catalyst of Example 1 is the same as that of Comparative Example 1 in terms of combination and production method, except the temperature rising condition toward the catalytic reaction temperature. That is, the catalyst was produced by the same method as the combination and the production steps of Comparative Example 1. Then, the reaction tube was charged therewith, followed by the carbonization treatment and then a temperature rising until 800° C. by 15 minutes while supplying Ar gas, a non-oxidizing gas, to this reaction tube.

### 2. Evaluation of Catalysts of Comparative Examples and Example

Evaluation method of the catalysts of Comparative Examples and Example is described. The baked body after the carbonization treatment to be put into the reaction tube shown in FIG. 5 was in 4.2 g (zeolite percentage 82.50%). Based on the reaction condition shown in Table 1, the catalyst was reacted with the reaction gas under conditions of a reaction space velocity=10000 ml/g−MFI/h (CH₄ gas flow base), a reaction temperature of 800° C., a reaction time of 15 hours, a reaction pressure of 0.3 MPa, while supplying a carbonic acid gas-mixed methane gas (the molar ratio of methane and carbonic acid gas, methane:carbonic acid gas (carbon dioxide), was 20:1) as a reaction gas to the fixed bed flow type reaction apparatus 1.

TABLE 1

| | Reaction Condition | |
|---|---|---|
| Catalyst Charge Amount | 4.2 | g |
| Zeolite Percentage | 82.50% | |
| Supply Gas | 100 Methane (CH₄) + 5 Carbonic | |
| | Acid Gas (CO₂) | |
| Reaction SV | 10000 ml/g - MFI/h (CH₄ gas flow base) | |
| Reaction Temperature | 800° | C. |
| Reaction Time | 15 | Hours |
| Reaction Pressure | 0.3 | MPa |

Upon this, analysis of the product was conducted. Based on the analysis result, methane conversion, benzene yield, naphthalene yield and BTX yield were examined over time. The product analysis was conducted by using TCD-GC and FID-GC.

Methane conversion, benzene yield, naphthalene yield and BTX yield are defined as follows.

Methane Conversion (%)=[(the amount of methane consumed in the methane reforming reaction)/(the amount of methane used for the methane reforming reaction)]×100

Benzene Yield (%)=[(the amount of benzene produced)/(the amount of methane used for the methane reforming reaction)]×100

Naphthalene Yield (%)=[(the amount of naphthalene produced)/(the amount of methane used for the methane reforming reaction)]×100

BTX Yield (%)=[(the amounts of benzene, toluene and xylene produced)/(the amount of methane used for the methane reforming reaction)]1×100

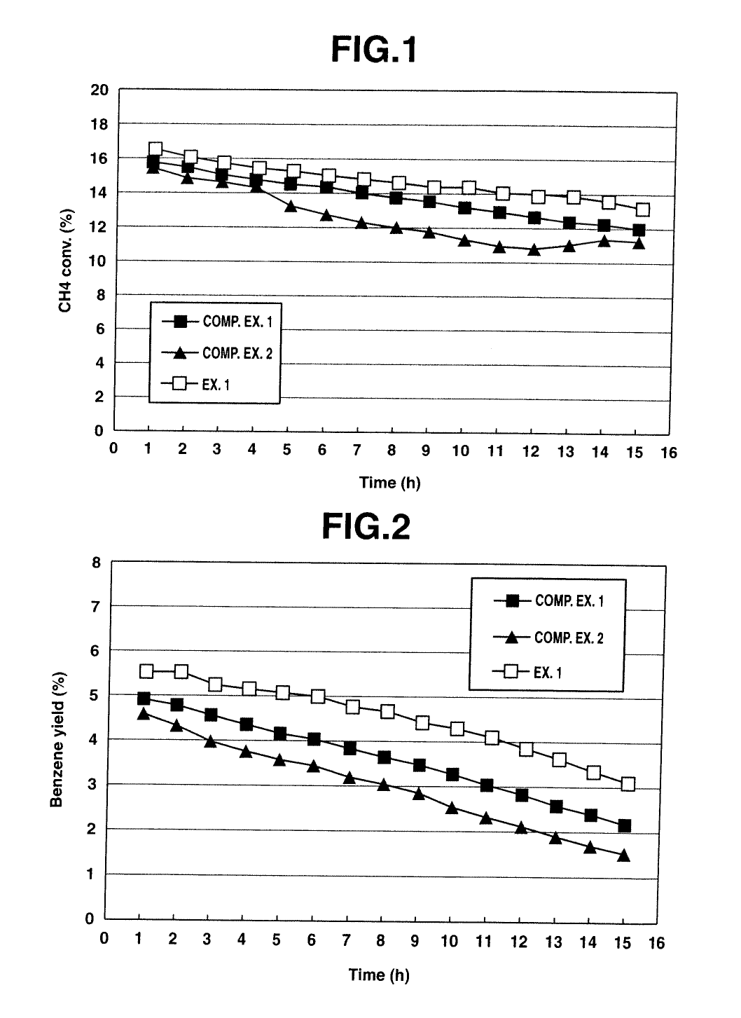

FIG. 1 shows a change over time of methane conversion in case that each catalyst of Comparative Example 1, Comparative Example 2 and Example 1 has been reacted with the carbonic acid gas-mixed methane gas. As is clear from FIG. 1, it is understood that stability over time of methane conversion is improved by conducting a temperature rising until the catalytic reaction temperature under the condition of Example 1, as compared with the cases of conducting a temperature rising under conditions of Comparative Example 1 and Comparative Example 2.

FIG. 2 shows a change over time of benzene yield in case that each catalyst of Comparative Example 1, Comparative Example 2 and Example 1 has been reacted with the carbonic acid gas-mixed methane gas. As is clear from FIG. 2, it is understood that stability over time of benzene yield is improved by conducting a temperature rising until the catalytic reaction temperature under the condition of Example 1, as compared with the cases of conducting temperature risings under conditions of Comparative Example 1 and Comparative Example 2.

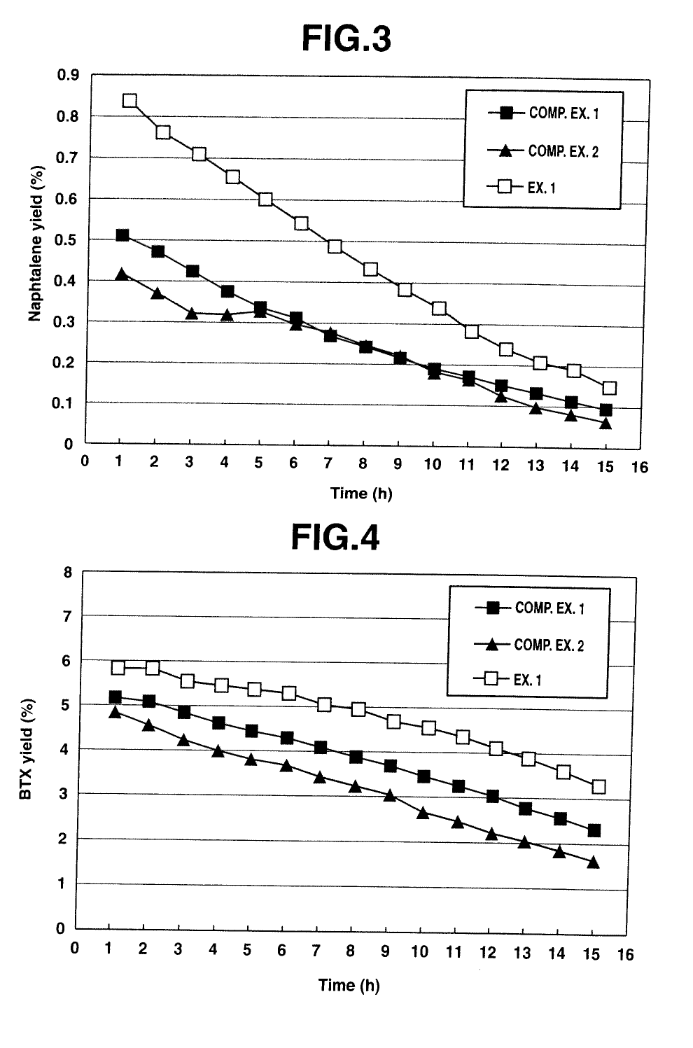

FIG. 3 shows a change over time of naphthalene yield in case that each catalyst of Comparative Example 1, Comparative Example 2 and Example 1 has been reacted with the carbonic acid gas-mixed methane gas. As is clear from FIG. 3, it is understood that stability over time of naphthalene yield is improved by conducting a temperature rising until the catalytic reaction temperature under the condition of Example 1, as compared with the cases of conducting temperature risings under conditions of Comparative Example 1 and Comparative Example 2.

FIG. 4 shows a change over time of BTX yield in case that each catalyst of Comparative Example 1, Comparative Example 2 and Example 1 has been reacted with the carbonic acid gas-mixed methane gas. As is clear from FIG. 4, it is understood that stability over time of BTX yield is improved by conducting a temperature rising until the catalytic reaction temperature under the condition of Example 1, as compared with the cases of conducting temperature risings under conditions of Comparative Example 1 and Comparative Example 2.

The condition of molybdenum carbide in the catalyst at the start of the catalytic reaction is reflected in the above results. Molybdenum carbide produced by the carbonization treatment is considered to be an active metal of a direct reaction to aromatic compounds and hydrogen. In Example 1, the catalyst is subjected to a temperature rising until the catalytic reaction temperature under a non-oxidizing gas atmosphere. With this, it is possible to stably maintain the condition of molybdenum carbide. Therefore, activity lifetime stability is improved.

On the other hand, a carbonic acid gas-mixed gas is allowed to flow upon temperature rising in Comparative Examples 1 and 2. At 700° C. or higher, molybdenum carbide is easily oxidized by carbon dioxide, which is an oxidizing gas, thereby turning into molybdenum oxide. That is, in Comparative Examples 1 and 2, the active species decreases upon temperature rising, and therefore the active lifetime stability is lowered. Furthermore, the active lifetime stability is more lowered in Comparative Example 2. This is because the period of time of flow of the carbonic acid gas-mixed gas was long to increase the period of time of contact between the oxidizing gas and the catalyst to allow the oxidation reaction of molybdenum carbide as an active species to proceed.

As mentioned above, according to the present invention, in a lower-hydrocarbon aromatizing catalyst prepared by supporting molybdenum on metallosilicate and then conducting a carbonization treatment of molybdenum, a temperature rising until the catalytic reaction temperature under a non-oxidizing gas atmosphere makes it possible to improve stability over time of methane conversion and to improve benzene yield, naphthalene yield, and yield of BTX, which are useful components such as benzene and toluene.

In the above-mentioned example, ZSM-5 is used as a metallosilicate on which a metal component is supported, but an effect similar to that of the above example is achieved even if MCM-22 is applied. Furthermore, the metal supported on metallosilicate is not limited to molybdenum and molybdenum compounds, and a metal known in conventional techniques may be supported thereon. Furthermore, in the above example, the amount of molybdenum supported is 6 wt % relative to the total amount of the catalyst after baking, but an effect similar to that of the above example is achieved if the amount of supporting is in a range of 2-12 wt % relative to the total amount of the catalyst.

Furthermore, in the above-mentioned example, the invention is conducted in a series of processes from the carbonization to the temperature rising toward the catalytic reaction temperature. However, the embodiment is not limited to this. A similar effect is achieved, even if a catalyst is separately prepared by previously conducting a carbonization, and the present invention is conducted when the catalyst after the carbonization treatment is subjected to a temperature rising from room temperature to the reaction temperature.

The non-oxidizing gas is preferably nitrogen, argon or helium. Flow rate of the gas is not particularly limited. Upon conducting a temperature rising until the catalytic reaction temperature, it suffices to conduct the temperature rising with a flow or substitution of the non-oxidizing gas.

Herein, in the evaluation method of the above example to produce aromatic compounds, it is reacted with a reaction gas in which a molar ratio of methane to carbonic acid gas, methane:carbonic acid gas (carbon dioxide), is 20:1, but an effect similar to that of the above-mentioned example is achieved even if the amount of the carbonic acid gas added is in a range of 0.5-6% relative to the total of the reaction gas.

1. In a method for producing an aromatic compound by a catalytic reaction using a lower hydrocarbon as a raw material,
the method for producing an aromatic compound using a lower hydrocarbon as a raw material being characterized in:
that a catalyst used in the catalytic reaction is subjected to a temperature rising until a catalytic reaction temperature under an atmosphere of a non-oxidizing gas except hydrocarbon gas and
that a gas containing a lower hydrocarbon is brought into contact with the catalyst to produce an aromatic compound.

2. A method for producing an aromatic compound using a lower hydrocarbon as a raw material according to claim 1, which is characterized in that the non-oxidizing gas is a reducing gas or inert gas.

3. A method for producing an aromatic compound according to claim 1, which is characterized in that the catalyst is a catalyst prepared by supporting molybdenum or a molybdenum compound on a metallosilicate and then conducting a carbonization treatment.
